(19) United States
(12) Patent Application Publication
(10) Pub. No.: US 2026/0176575 A1
(43) Pub. Date: Jun. 25, 2026

(54) MAGNETOSPIRILLUM GRYPHISWALDENSE STRAIN MSR-1, CULTURE MEDIUM, AND USE THEREOF

(71) Applicant: Institute of Soil Science, Chinese Academy of Sciences, Nanjing (CN)

(72) Inventors: Chen TU, Nanjing (CN); Yongming LUO, Nanjing (CN); Junchao HAN, Nanjing (CN); Zhenyu CAO, Nanjing (CN); Ying LIU, Nanjing (CN)

(21) Appl. No.: 19/317,917

(22) Filed: Sep. 3, 2025

(30) Foreign Application Priority Data
Dec. 23, 2024 (CN) 202411895810.8

(51) Int. Cl. C12N 1/20; B09C 1/10; C02F 3/34; C02F 101/20; C09K 17/00; C12R 1/01

(52) U.S. Cl. CPC C12N 1/205; B09C 1/10; C02F 3/34; C09K 17/00; C02F 2101/20; C12R 2001/01

(57) ABSTRACT

A Magnetospirillum gryphiswaldense strain MSR-1, and a culture method and use thereof are provided, belonging to the technical field of environmental microorganisms. Provided is a Magnetospirillum gryphiswaldense strain MSR-1 with a deposit number of CCTCC NO: M 20242494. Conditions for culturing the Magnetospirillum gryphiswaldense strain MSR-1 are optimized, including adjusting key factors such as different carbon sources, different nitrogen sources, different iron sources, and different reducing agents, thereby simultaneously improving a biomass and a magnetic production activity of the strain MSR-1. Provided is use of the Magnetospirillum gryphiswaldense strain MSR-1 in bioremediation of cadmium polluted water and soil, thereby providing a new idea for solving the increasingly serious environmental problem of heavy metal pollution.

### CROSS REFERENCE TO RELATED APPLICATION

This patent application claims the benefit and priority of Chinese Patent Application No. 202411895810.8 filed with the China National Intellectual Property Administration on Dec. 23, 2024, the disclosure of which is incorporated by reference herein in its entirety as part of the present application.

### TECHNICAL FIELD

The present disclosure belongs to the technical field of environmental microorganisms, and specifically relates to a Magnetospirillum gryphiswaldense strain MSR-1, and a medium, a culture method, and use thereof.

### BACKGROUND

Heavy metal pollution in the environment comes from a wide range of sources, including wastewater discharge from metal smelting, mineral mining, electronic product production, electroplating, and chemical industries, as well as sewage and sludge irrigation, and the application of insecticides, pesticides, and fertilizers. Among various heavy metal pollution, the pollution of cadmium (Cd) ranks first. According to the “National Soil Pollution Status Survey Bulletin”, cadmium has the maximum rate of exceeding the standard, leading to cadmium pollution in over 40% of multiple heavy metal polluted soil. Cadmium is one of the most easily known harmful substances that accumulate in organisms, with a half-life of 10 to 30 years. Human activities have led to an increasing amount and an increasingly wider distribution range of cadmium in the environment. Once entering the natural environment, heavy metal pollutants can be absorbed by animals and plants and then gradually accumulate in the organs and tissues of animals and the roots, stems, leaves, and fruits of plants, eventually affecting human health through the food chain. Therefore, remediation of cadmium-contaminated water and soil is of great significance for protecting the ecological environment and human health.

At present, the remediation methods for heavy metal pollution in soil and water environments at home and abroad are mainly divided into three categories. The first is physical remediation, such as removing topsoil, soil dressing, and leaching. This remediation method can quickly achieve stable remediation. However, there are also obvious shortcomings including a lot of financial and human resources, making the method not suitable for large-scale polluted area. The second is chemical remediation, which is to add some chemical additives into the soil to change the form of heavy metals in the soil, thereby reducing their bioavailability and biological effectiveness. Such a remediation method has a moderate control effect on cadmium pollution, but cadmium may be re-activated once environmental conditions change. The third is bioremediation, which mainly covers various forms such as phytoremediation and microbial remediation. The bioremediation is to absorb, immobilize, transform, and uptake heavy metal pollution through certain characteristics of the organisms themselves. Bioremediation has become a research hotspot in the field of soil heavy metal remediation since it adheres to the concepts of ecological and sustainable. Compared with traditional physical-chemical remediation, bioremediation is more suitable for in-situ remediation of non-point source heavy metal polluted soil. Moreover, this remediation method has a series of advantages such as low cost, environmental friendliness, simple operation, and low secondary pollution. Microbial remediation has received widespread attention. Microorganisms can remove heavy metals from the environment or transform them into less toxic forms through processes such as adsorption, enrichment, and transformation. However, it is still a huge challenge to separate and recover these microorganisms with specific functions from environmental media.

At present, the research on using microorganisms as a remediation agent for environmental heavy metal pollution control has already laid a certain foundation at home and abroad. Magnetotactic bacterium is a general term for a class of microorganisms that can move along the direction of a magnetic field. There are many types of magnetotactic bacteria in nature. There have been studies that remove heavy metals such as Ag and Cu from water bodies using the properties of magnetotactic bacteria. However, there is few public use of an MSR-1 strain of the magnetotactic bacteria for remediation of cadmium pollution in water or soil.

### SUMMARY

In view of this, an objective of the present disclosure is to provide a Magnetospirillum gryphiswaldense strain MSR-1, and a medium, a culture method, and use thereof. In the present disclosure, the medium and culture conditions of the Magnetospirillum gryphiswaldense strain MSR-1 are optimized to simultaneously increase its biomass and magnetic production activity, thereby achieving remediation of water and soil polluted by heavy metal cadmium using same.

To achieve the above objective, the present disclosure provides the following technical solutions:

The present disclosure provides a Magnetospirillum gryphiswaldense strain MSR-1, where the Magnetospirillum gryphiswaldense strain MSR-1 is deposited with a deposit number of CCTCC NO: M 20242494.

The present disclosure provides a medium for culturing the Magnetospirillum gryphiswaldense strain MSR-1, where in one embodiments, the medium includes carbon, and the medium includes 2 g/L to 5 g/L of a carbon source, 0.2 g/L to 0.5 g/L of a nitrogen source, 0.005 g/L to 0.02 g/L of an iron source, and 0.03 g/L to 0.06 g/L of a reducing agent.

In some embodiments, the medium includes 3 g/L to 4 g/L of sodium lactate, 0.35 g/L to 0.50 g/L of ammonium chloride, 0.010 g/L to 0.015 g/L of ferric citrate, and 0.045 g/L to 0.060 g/L of sodium thioglycolate.

The present disclosure further provides a method for culturing the Magnetospirillum gryphiswaldense strain MSR-1, including the following steps: inoculating an activated Magnetospirillum gryphiswaldense strain MSR-1 into the medium, and then culturing at 25° C. to 35° C. under 90 r/min to 120 r/min for 18 h to 48 h.

In some embodiments, the culturing is conducted for 20 h to 36 h.

The present disclosure further provides a preparation product, including the Magnetospirillum gryphiswaldense strain MSR-1.

The present disclosure further provides use of the Magnetospirillum gryphiswaldense strain MSR-1 or the preparation product in remediation of cadmium pollution, including remediation of the cadmium pollution in water and/orsoil.

As an embodiment, the water or the soil is adjusted to a pH value of 5.0 to 8.0 and an initial cadmium concentration is no more than 2 mg/L during the remediation.

In the present disclosure, the Magnetospirillum gryphiswaldense strain MSR-1 is inoculated into the water at 3 g/L to 10 g/L; for soil treatment, a soil-water mixture is prepared, and the strain MSR-1 is inoculated into this mixture at a rate of 3% to 5% (w/w) relative to the soil mass.

Compared with the prior art, the present disclosure has the following beneficial effects:

The present disclosure provides a Magnetospirillum gryphiswaldense strain MSR-1, and a specialized culture medium, a cultivation method, and use thereof. In the present disclosure, the Magnetospirillum gryphiswaldense strain MSR-1 is taken as a core research object, and effects of key factors such as different types of carbon sources, nitrogen sources, iron sources, and reducing agents and addition amounts on the Magnetospirillum gryphiswaldense strain MSR-1 are deeply explored. A medium and culture conditions that can simultaneously improve a biomass and a magnetic production activity of the MSR-1 are designed through a large number of sophisticated experiments and data analysis, thereby achieving efficient utilization of the Magnetospirillum gryphiswaldense strain MSR-1. On this basis, the present disclosure provides a preparation product prepared by the Magnetospirillum gryphiswaldense strain MSR-1. The present disclosure further expands the application scope of the Magnetospirillum gryphiswaldense strain MSR-1 and the preparation product thereof in the field of heavy metal cadmium pollution control, and applies same to the practice of remediation of cadmium polluted water and soil. With the unique physiological characteristics of the strain MSR-1 and by regulating external environmental conditions, the preparation product can effectively adsorb and remove cadmium element in polluted water or soil with an initial cadmium concentration of 2 mg/L (or 2 mg/kg), achieving effective remediation of water and soil polluted by heavy metal cadmium. In addition, magnetic adsorption is conducted to achieve efficient recovery of the remediation agent, thus effectively avoiding the generation of secondary pollution. The present disclosure provides a high-efficiency, low-cost, and environmentally sustainable method for the bioremediation of cadmium-polluted water and soil, and is of great significance for solving the increasingly serious heavy metal pollution in the environment.

### BRIEF DESCRIPTION OF THE DRAWINGS

FIG. 1 shows a scanning electron microscopy (SEM) image of the Magnetospirillum gryphiswaldense strain MSR-1;

FIGS. 2A-2D show effects of different carbon sources, different nitrogen sources, different iron sources, and different reducing agents on the culturing of the Magnetospirillum gryphiswaldense strain MSR-1;

FIGS. 3A-3F show three dimensional response surface optimization reaction diagram of the interactive effects of different factors on the bacterial concentration of the strain MSR-1, where letters in FIGS. 4A-4F represent: A: dosage of sodium lactate; B: dosage of ammonium chloride; C: dosage of ferric citrate; D: dosage of sodium thioglycolate;

FIGS. 4A-4F show a three-dimensional response surface optimization reaction diagram of the interactive effects of different factors on a magnetosome yield of the strain MSR-1, where letters in FIGS. 4A-4F represent: A: dosage of sodium lactate; B: dosage of ammonium chloride; C: dosage of ferric citrate; D: dosage of sodium thioglycolate;

FIG. 5 shows a growth curve and a C_{mag} curve of the Magnetospirillum gryphiswaldense strain MSR-1 under the optimized conditions of response surface optimization;

FIGS. 6A-6D) shows effects of different pH values, initial Cd²⁺ concentrations, MSR-1 biomass concentrations, and temperatures on an efficiency of the Magnetospirillum gryphiswaldense strain MSR-1 in adsorbing Cd²⁺, where FIG. 6A: pH; FIG. 6B: initial Cd²⁺ concentration; FIG. 6C: MSR-1 biomass concentration; FIG. 6D: temperature;

FIGS. 7A-7B shows analysis of the adsorption kinetics and adsorption isotherms of Cd²⁺ by the Magnetospirillum gryphiswaldense strain MSR-1, where FIG. 7A is the analysis of adsorption kinetics, and FIG. 7B is the analysis of adsorption isotherms;

FIG. 8 shows an effect of the Magnetospirillum gryphiswaldense strain MSR-1 on removal of cadmium from cadmium-polluted water; and

FIG. 9 shows pollution reduction and remediation effects of the Magnetospirillum gryphiswaldense strain MSR-1 on different Cd-polluted soil samples, where * represents p<0.05, ** represents p<0.01.

### DEPOSIT OF BIOLOGICAL MATERIAL

The Magnetospirillum gryphiswaldense strain MSR-1 was deposited in the China Center for Type Culture Collection (CCTCC), No. 299, Bayi Road, Wuchang District, Wuhan City, Hubei Province on Nov. 8, 2024 with a Deposit number CCTCC NO: M 20242494

### DETAILED DESCRIPTION OF THE EMBODIMENTS

The present disclosure provides a Magnetospirillum gryphiswaldense strain MSR-1, where the Magnetospirillum gryphiswaldense strain MSR-1 is deposited with a deposit number of CCTCC NO: M 20242494. The Magnetospirillum gryphiswaldense strain MSR-1 belongs to the class α-Proteobacteria and originates from freshwater sediments.

The present disclosure provides a culture medium for the Magnetospirillum gryphiswaldense strain MSR-1. In the medium, a carbon source includes sodium lactate; a nitrogen source includes ammonium chloride or ammonium sulfate; an iron source includes one of ferrous sulfate, ferric citrate, ferric quinate, and compound iron (ferrous sulfate and ferric citrate compounded in a ratio of 1:1); and a reducing agent includes one of sodium thioglycolate, ascorbic acid, and cysteine. In present disclosure, an activated Magnetospirillum gryphiswaldense strain MSR-1 is inoculated and cultured into media containing different types of carbon sources, nitrogen sources, iron sources, and reducing agents to screen the optimal conditions for biological status and magnetic production ability of the Magnetospirillum gryphiswaldense strain MSR-1. In some embodiments, the carbon source is sodium lactate. In some embodiments, the nitrogen source is ammonium chloride. In some embodiments, the iron source is ferric citrate. In some embodiments, the reducing agent is sodium thioglycolate. The dosages of different types of carbon sources, nitrogen sources, iron sources, and reducing agents are also considered. Specifically, the medium includes 2 g/L to 5 g/L of the carbon source, 0.2 g/L to 0.5 g/L of the nitrogen source, 0.005 g/L to 0.02 g/L of the iron source, and 0.03 g/L to 0.06 g/L of the reducing agent. In a preferred medium, the carbon source is sodium lactate, with a dosage of 2 g/L to 5 g/L; the nitrogen source is ammonium chloride, with a dosage of 0.2 g/L to 0.5 g/L; the iron source is ferric citrate, with a dosage of 0.005 g/L to 0.02 g/L; and the reducing agent is sodium thioglycolate, with a dosage of 0.03 g/L to 0.06 g/L. In some other embodiments, the medium includes 3 g/L to 4 g/L of sodium lactate, 0.35 g/L to 0.50 g/L of ammonium chloride, 0.010 g/L to 0.015 g/L of ferric citrate, and 0.045 g/L to 0.060 g/L of sodium thioglycolate. In some other embodiments, the medium includes 3.38 g/L of the sodium lactate, 0.50 g/L of the ammonium chloride, 0.011 g/L of the ferric citrate, and 0.060 g/L of the sodium thioglycolate.

The present disclosure further provides a culture method of the Magnetospirillum gryphiswaldense strain MSR-1, including the following steps: inoculating an activated Magnetospirillum gryphiswaldense strain MSR-1 into the medium containing the carbon source, the nitrogen source, the iron source, and the reducing agent, and then culturing at 25° C. to 35° C. under 90 r/min to 120 r/min for 18 h to 48 h. An activation process includes: taking out the Magnetospirillum gryphiswaldense strain MSR-1 from a frozen glycerol stock, inoculating into a magnetotactic bacterium liquid medium, and then culturing in a shaker at 30° C. under 110 r/min for 24 h until an OD₅₆₅ value reaches 0.6. The magnetotactic bacteria liquid medium includes 0.01 g/L to 0.02 g/L of ferric citrate, 0.05 g/L to 0.15 g/L of yeast powder, 0.30 g/L to 0.60 g/L of potassium dihydrogen phosphate, 0.30 g/L to 0.50 g/L of ammonium chloride, 0.05 g/L to 0.20 g/L of magnesium sulfate, 2.5 g/L to 4.0 g/L of sodium lactate, 0.04 g/L to 0.07 g/L of sodium thioglycolate, and 0.005% to 0.2% of trace elements. The trace elements include 1.20 g/L to 1.70 g/L of nitrilotriacetic acid, 2.6 g/L to 3.2 g/L of MgSO_{4·}7H₂O, 0.3 g/L to 0.7 g/L of MnSO₄·H₂O, 0.8 g/L to 1.3 g/L of NaCl, 0.05 g/L to 0.14 g/L of FeSO_{4·}7H₂O, 0.05 g/L to 0.14 g/L of CoCl₂·6H₂O, 0.05 g/L to 0.14 g/L of CaCl₂), 0.05 g/L to 0.14 g/L of ZnSO_{4·}7H₂O, 0.0080 g/L to 0.014 g/L of CuSO₄·5H₂O, 0.0080 g/L to 0.014 g/L of AIK(SO₄)₂·12H₂O, 0.0080 g/L to 0.014 g/L of H₃BO₃, and 0.0080 g/L to 0.013 g/L of Na₂MoO₄·2H₂O. In some embodiments, the trace elements include 1.5 g/L of nitrilotriacetic acid, 3.0 g/L of MgSO_{4·}7H₂O, 0.5 g/L of MnSO₄·H₂O, 1.0 g/L of NaCl, 0.1 g/L of FeSO_{4·}7H₂O, 0.1 g/L of CoCl₂·6H₂O, 0.1 g/L of CaCl₂), 0.1 g/L of ZnSO_{4·}7H₂O, 0.01 g/L of CuSO_{4·}5H₂O, 0.01 g/L of AIK(SO₄)_{2·}12H₂O, 0.01 g/L of H₃BO₃, and 0.01 g/L of Na₂MoO₄·2H₂O. In some embodiments, the magnetotactic bacteria liquid medium includes 0.01 g/L of ferric citrate, 0.10 g/L of yeast powder, 0.5 g/L of potassium dihydrogen phosphate, 0.4 g/L of ammonium chloride, 0.10 g/L of magnesium sulfate, 3 g/L of sodium lactate, 0.05 g/L of sodium thioglycolate, and 0.1% of trace elements. In some embodiments, a method for preparing the magnetotactic bacteria liquid medium includes adding the above-mentioned various components into deionized water, adjusting the pH value, and then autoclaving. An acid used to adjust the pH value includes one or more of dilute hydrochloric acid, phosphoric acid, and citric acid. In some embodiments, the dilute hydrochloric acid is added to adjust the pH value. The dilute hydrochloric acid has a concentration of 0.5 mol/L to 2 mol/L. In some other embodiments, the dilute hydrochloric acid has a concentration of 1 mol/L. The pH value is 6 to 8. In some embodiments, the pH value is 6 to 7. In some other embodiments, the pH value is 6.98. The optimized Magnetospirillum gryphiswaldense strain MSR-1 is cultured in a shaker at 25° C. to 35° C. under 90 r/min to 120 r/min for 18 h to 48 h. In some embodiments, the culturing is conducted in a shaker at 30° C. under 110 r/min. In some embodiments, the culturing is conducted for 20 h to 36 h. In some other embodiments, the culturing is conducted for 24 h. By optimizing and adjusting the above key factors, the optimal conditions are determined for the biological status and magnetic production capacity of the Magnetospirillum gryphiswaldense strain MSR-1.

The present disclosure further provides a preparation product, including the Magnetospirillum gryphiswaldense strain MSR-1 with a deposit number of CCTCC NO: M 20242494. In some embodiments of the present disclosure, the preparation product further includes an auxiliary material. There is no special limitation on a type of the auxiliary material, and any auxiliary material of biological preparations known in the art can be used, such as water or magnetotactic bacterial liquid medium. There is no particular limitation on a method for preparing the preparation product, and a preparation scheme for bacterial products known in the art may be used, such as mixing a bacterial solution of the Magnetospirillum gryphiswaldense strain MSR-1 after amplification culture with the auxiliary material.

The present disclosure further provides use of the Magnetospirillum gryphiswaldense strain MSR-1 or the preparation product containing the Magnetospirillum gryphiswaldense strain MSR-1 in remediation of cadmium pollution, including remediation of the cadmium pollution in water and/or soil. When the Magnetospirillum gryphiswaldense strain MSR-1 or the preparation product containing the Magnetospirillum gryphiswaldense strain MSR-1 is used, a pH value of the water or the soil should be adjusted to 5.0 to 8.0. In some embodiments, the pH value is 8.0. A reagent for adjusting the pH value may be one or more of hydrochloric acid, citric acid, sodium hydroxide, and sodium bicarbonate, but is not limited to the above. Higher concentrations of heavy metal cadmium can be toxic to bacteria and cause bacterial cell damage, such that an initial concentration of cadmium polluted water needs to be set. The initial concentration of cadmium in cadmium polluted water is set to 0 mg/L to 5 mg/L. In some embodiments, the initial cadmium concentration is 0 mg/L to 2 mg/L. In some other embodiments, the initial cadmium concentration is 0.5 mg/L to 2 mg/L. In some other embodiments, the initial cadmium concentration is 2 mg/L. In the application of remediation of the cadmium pollution, the Magnetospirillum gryphiswaldense strain MSR-1 is inoculated into the water at 3 g/L to 10 g/L. In some embodiments, the Magnetospirillum gryphiswaldense strain MSR-1 is inoculated into the water at 3 g/L to 7 g/L. In some other embodiments, the Magnetospirillum gryphiswaldense strain MSR-1 is inoculated into the water at 3 g/L to 4 g/L. The use in remediation of cadmium pollution in water specifically includes the following steps: detecting an initial cadmium concentration of polluted water, and adjusting the initial cadmium concentration of the polluted water to maintain at 0 mg/L to 5 mg/L; adjusting the pH value and temperature of the polluted water and inoculating the Magnetospirillum gryphiswaldense strain MSR-1 or the preparation product into the polluted water at 3 g/L to 10 g/L, and stirring the inoculated polluted water evenly and then allowing to stand for 1 h to 2 h. A magnetic field is applied outside the polluted water to allow magnetic adsorption, such that sewage is separated from bacterial cells without disturbing the bacterial cells magnetically enriched on a side wall, thereby achieving the removal of heavy metal cadmium in the sewage. The temperature is 20° C. to 37° C. In some embodiments, temperature is 30° C. After inoculation of Magnetospirillum gryphiswaldense, the polluted water is allowed to stand for 1 h to 2 h. In some embodiments, the polluted water is allowed to stand for 1 h. The magnetic adsorption is conducted for 18 h to 30 h. The present disclosure further provides use in remediation of cadmium pollution in soil, including the following steps: crushing and grinding cadmium polluted soil, adding the soil and water according to a mass ratio, stirring evenly to obtain a water-soil mixture; inoculating the Magnetospirillum gryphiswaldense strain MSR-1 or the preparation product into the water-soil mixture at 3% to 15% of a soil mass, stirring an inoculated water-soil mixture evenly, and allowing to stand for 1 h to 2 h; applying a magnetic field outside the water-soil mixture to allow magnetic adsorption, such that water, soil, and bacterial cells are separated without disturbing the bacterial cells magnetically enriched on a side wall, thereby achieving the removal of heavy metal cadmium in the soil. The soil and the water are at a mass ratio of 1: (8-12). In some embodiments, the soil and the water are at a mass ratio of 1:10. In some embodiments, a mass concentration of cadmium in the soil is detected by acid digestion of the soil, and soil samples are digested with a graphite furnace digester (SPH-3, Yuanxi Instrument, Shanghai); there is no special restriction on a digestion procedure and the like, and the schemes well known in the art can be adopted. The mass concentration of cadmium is detected by inductively coupled plasma-mass spectrometry (ICP-MS) (PE Nexion 2000, USA); there is no special limitation on a detection method, and any method known in the art can be used.

In the present disclosure, the Magnetospirillum gryphiswaldense strain MSR-1 or a preparation product containing the Magnetospirillum gryphiswaldense strain MSR-1 is applied to the remediation of cadmium polluted. Taking anhydrous CdCl₂ with different mass concentrations to simulate cadmium-containing industrial wastewater, cadmium-containing soil in a certain place in Changsha, Hunan, and cadmium-containing soil in a certain place in Baiyin, Gansu as examples, the cadmium removal capacity of the Magnetospirillum gryphiswaldense strain MSR-1 is verified. The results show that the Magnetospirillum gryphiswaldense strain MSR-1 or a strain-containing preparation product thereof can effectively reduce the content of cadmium ions in water, and a cadmium removal efficiency in water can reach 92.2%, showing excellent remediation potential for cadmium removal and reduction. When remediating cadmium pollution in the soil, the Magnetospirillum gryphiswaldense strain MSR-1 or a strain-containing preparation product thereof can reduce the total cadmium content in the soil of Changsha and Baiyin by 12.7% and 15.6%, respectively, also showing high remediation potential for cadmium removal and reduction.

In the present disclosure, the data are sorted and analyzed using Microsoft Excel 2019, and one-way analysis of variance (ANOVA) is conducted using SPSS 27.0. P<0.05 is defined as a significant difference, and Origin 2023 software was used to allow plotting.

In the following examples, all methods are conventional methods, unless otherwise specified.

The materials, reagents, and the like used in the following examples are all commercially available, unless otherwise specified.

The technical solution provided by the present disclosure will be described in detail below with reference to the accompanying drawings and examples, but they should not be construed as limiting the claimed scope of the present disclosure.

### Example 1

Surface Morphology of Magnetospirillum gryphiswaldense Strain MSR-1

A bacterial solution was centrifuged at 3,040 g for 15 min, a supernatant was discarded to obtain a bacterial pellet, 2.5% glutaraldehyde solution was added into the bacterial pellet and fixed at 4° C. for 12 h, then washed 2 times with 0.01 mol/L sterile PBS buffer, and the washed bacteria were placed sequentially in 50%, 75%, 90%, and 100% ethanol solutions to allow dehydration for 15 min. The bacterial cells were freeze-dried and observed using SEM (Zeiss sigma300, Zeiss, Germany).

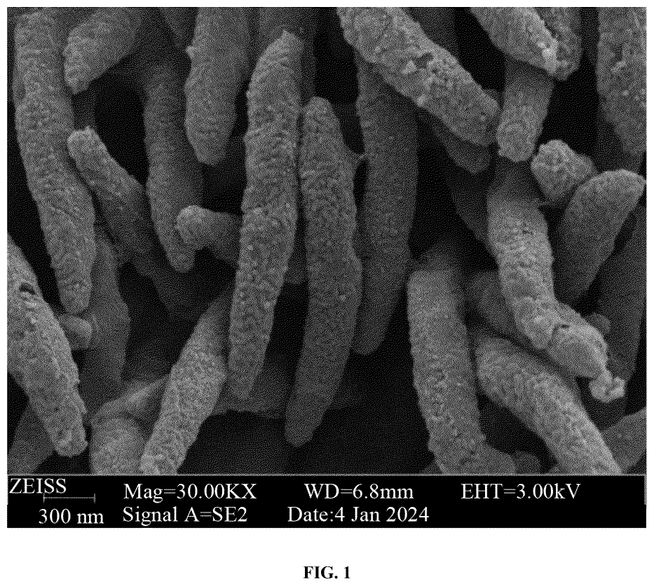

As shown in FIG. 1, the Magnetospirillum gryphiswaldense strain MSR-1 cells were spiral-shaped, with a size of approximately 2.0×0.3 μm, and an intact cell surface.

### Example 2

Optimization of Conditions for Cultivation of Magnetospirillum gryphiswaldense Strain MSR-1 and Magnetic Production
1.1 Optimization of Single Factor Conditions for Magnetospirillum gryphiswaldense Strain MSR-1

The Magnetospirillum gryphiswaldense strain MSR-1 was taken out from the frozen glycerol stock, activated and cultured in magnetotactic bacteria liquid medium, and cultured in a shaker (110 r/min, 30° C.) for 24 h. The bacterial cells were inoculated into media with different carbon sources (sodium acetate, tartaric acid, succinic acid, and sodium lactate), different nitrogen sources (sodium nitrate, ammonium chloride, and ammonium sulfate), different iron sources (ferrous carbonate, ferrous sulfate, ferric citrate, ferric quinate, and compound iron), and different reducing agents (sodium thioglycolate, ascorbic acid, and cysteine), and cultured in a shaker at 30° C. under 110 r/min for 24 h, and the growth status (OD₅₆₅ₙₘ) and magnetic production capacity (C_{mag}) were measured. To determine the optimal formulation of the required nutrients, three replicates were set up for each treatment.

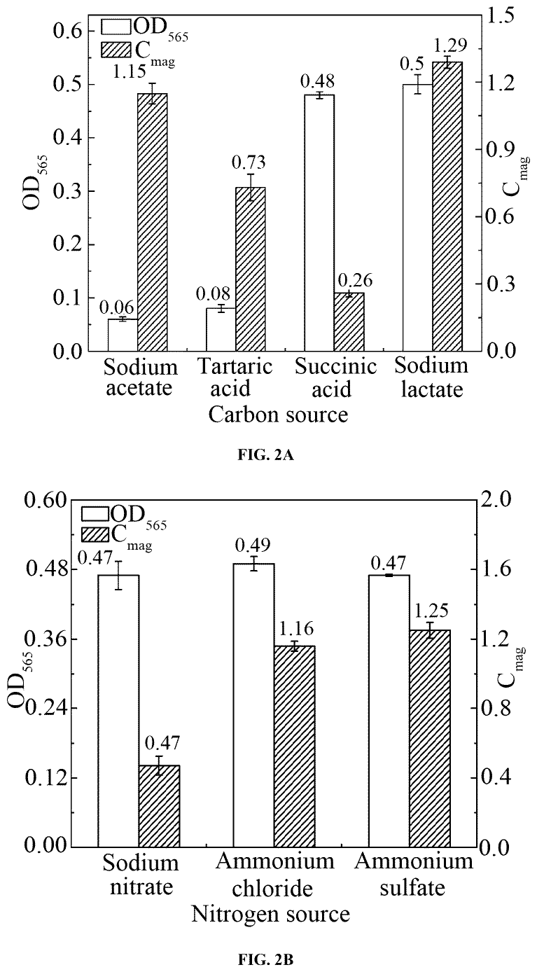

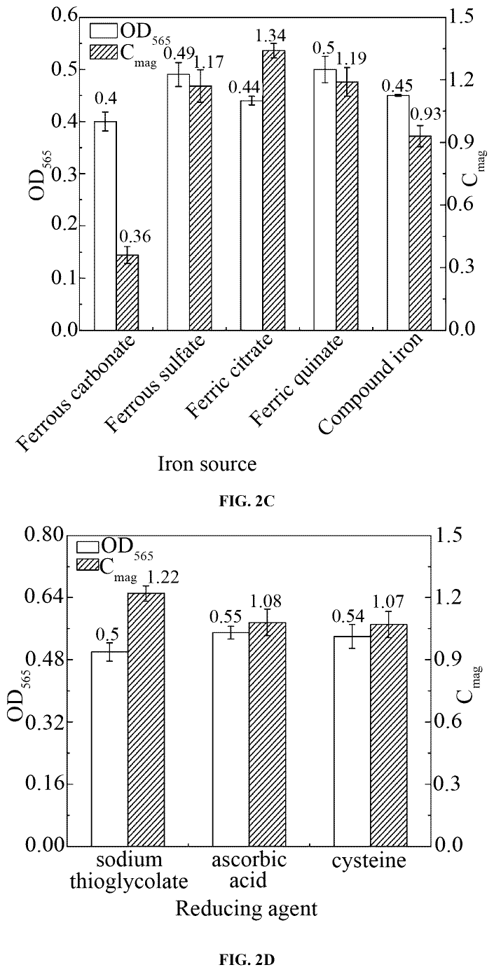

As shown in FIGS. 2A-2D, the Magnetospirillum gryphiswaldense strain MSR-1 had an optimal carbon source of sodium lactate, an optimal nitrogen source of ammonium chloride, an optimal iron source of ferric citrate, and an optimal reducing agent of sodium thioglycolate for growth and magnetic production.

1.2 Optimization of Single Factor Dosage of Magnetospirillum gryphiswaldense Strain MSR-1

According to the results under “1.1 Optimization of single factor conditions for Magnetospirillum gryphiswaldense strain MSR-1”, the optimal formula was determined, and different carbon source concentrations (1, 2, 3, 4, and 5 g/L), different nitrogen source concentrations (0.1, 0.2, 0.3, 0.4, and 0.5 g/L), different iron source concentrations (0, 0.005, 0.010, 0.015, and 0.020 g/L), and different reducing agent concentrations (0.02, 0.03, 0.04, 0.05, and 0.06 g/L) were set, respectively. The bacterial cells were cultured in a shaker at 30° C. under 110 r/min for 24 h, and the growth conditions (OD₅₆₅ₙₘ) and magnetic production capacity (C_{mag}) were measured. To determine the optimal dosage of the required nutrients, three replicates were set up for each treatment. The value of C_{mag} was obtained by adding a two-dimensional Helmholtz coil to the UV spectrophotometer, recording the OD₅₆₅ₙₘ of the bacterial solution when the magnetic field was parallel to the light path and perpendicular to the light path, which were recorded as OD_{⊥}(OD parallel) and OD_{⊥}(OD perpendicular), respectively, and calculating C_{mag} by calculating the ratio of OD_{⊥}and OD_{⊥}.

As shown in FIGS. 2A-2D, the best carbon source suitable for the growth and magnetic production of Magnetospirillum gryphiswaldense strain MSR-1 was sodium lactate, with a dosage of 2 g/L to 5 g/L. The best nitrogen source was ammonium chloride, with a dosage of 0.2 g/L to 0.5 g/L. The best iron source was ferric citrate, with a dosage of 0.005 g/L to 0.02 g/L. The best reducing agent was sodium thioglycolate, with a dosage of 0.03 g/L to 0.06 g/L.

1.3 Optimization of Culture Conditions of Magnetospirillum gryphiswaldense Strain MSR-1 by Response Surface Methodology

Taking Magnetospirillum gryphiswaldense strain MSR-1 as a tested strain, according to the results under “1.1” and “1.2”, a response surface experiment was designed with the dosages of carbon source, the nitrogen source, the iron source, and the reducing agent as four factors, and the growth condition (OD₅₆₅ₙₘ) and magnetic production capacity (C_{mag}) as two response values. The optimal conditions for growth and magnetic production were optimized, and the reliability of results was verified by repeated experiments.

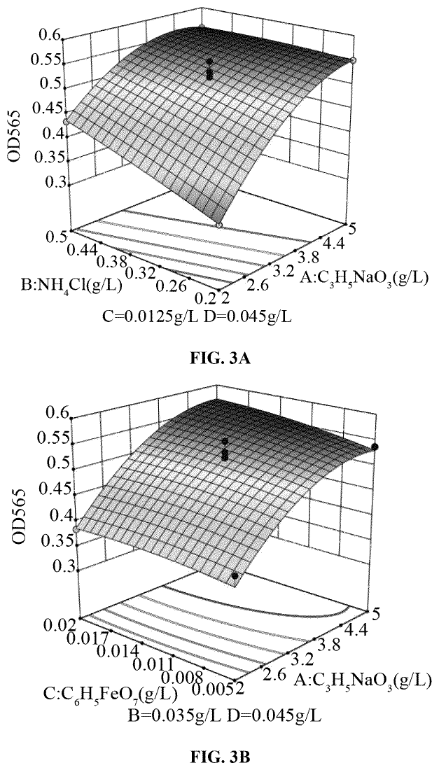

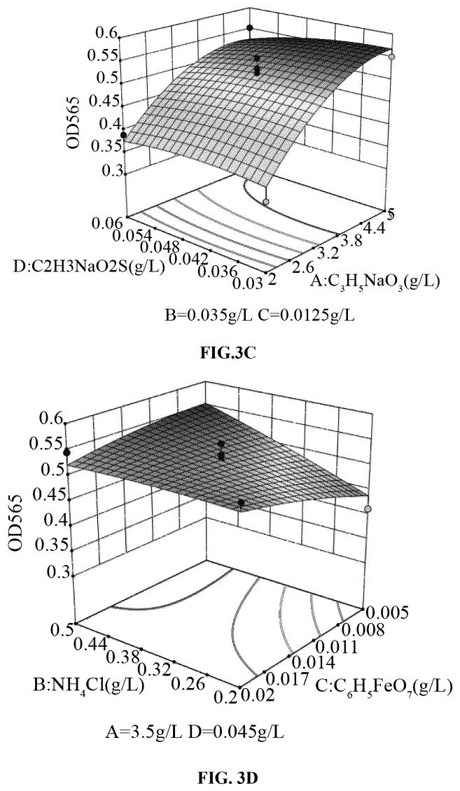

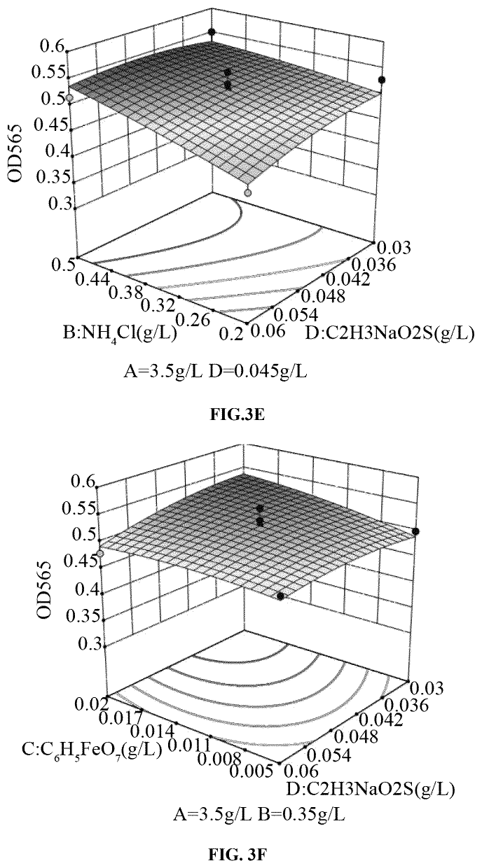

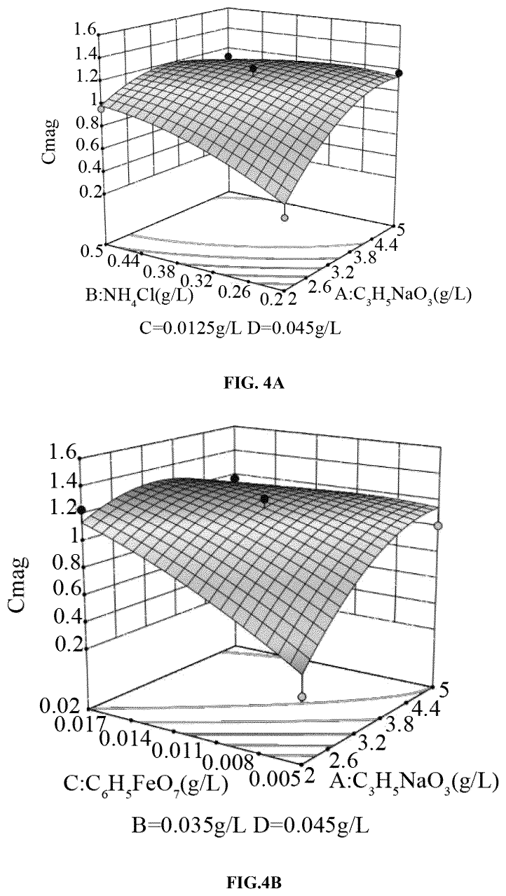

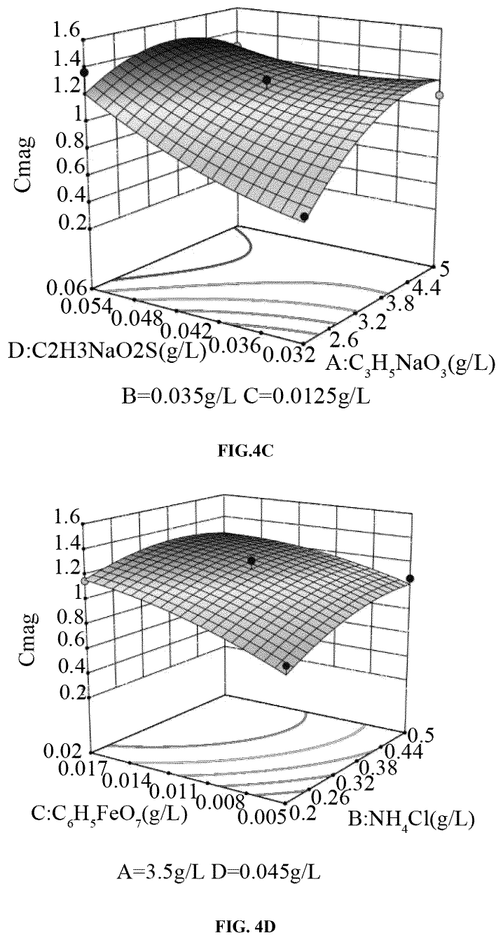

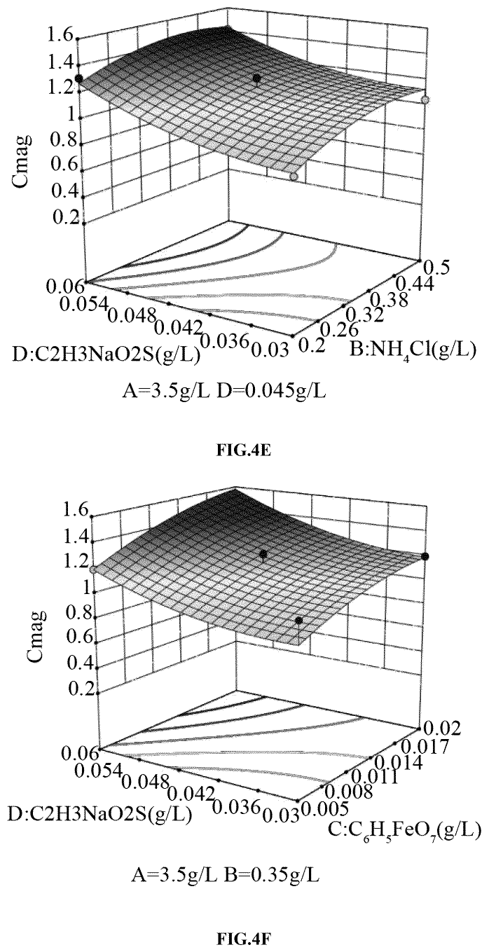

As shown in FIGS. 3A-3F and FIGS. 4A-4F, the growth and magnetic production capacity of the Magnetospirillum gryphiswaldense strain MSR-1 were optimal when the dosages of sodium lactate, ammonium chloride, ferric citrate, and sodium thioglycolate were 3.38 g/L, 0.50 g/L, 0.011 g/L, and 0.06 g/L, respectively. The optimization results showed that in the best case, OD 565 nm was 0.548 and C_{mag} was 1.38. The strain was cultured according to the theoretical values. In practice, the repeated culture was carried out at this case, and the strain OD₅₆₅ₙₘ was stabilized above 0.5 and C_{mag} was stabilized above 1.3, confirming the reliability of the experimental verification on the dosages of sodium lactate, ammonium chloride, ferric citrate, and sodium thioglycolate.

1.4 Time for Culture Magnetospirillum gryphiswaldense Strain MSR-1

The Magnetospirillum gryphiswaldense strain MSR-1 was taken out from the frozen glycerol stock and cultured for 0, 4, 8, 12, 16, 24, 30, 36, and 48 h separately, and the OD₅₆₅ₙₘ and C_{mag} of the bacterial solution were measured. The wavelength of the UV spectrophotometer was adjusted to 565 nm and an uninoculated medium was used as a blank for zero setting. 2.5 mL of the bacterial solution of Magnetospirillum gryphiswaldense strain MSR-1 was injected into a quartz cuvette for measurement, and 3 replicates were set at each time point. The growth curve of the optimized Magnetospirillum gryphiswaldense strain MSR-1 and the dynamic synthesis of magnetosomes were plotted using an UV spectrophotometer and a magnetic response instrument.

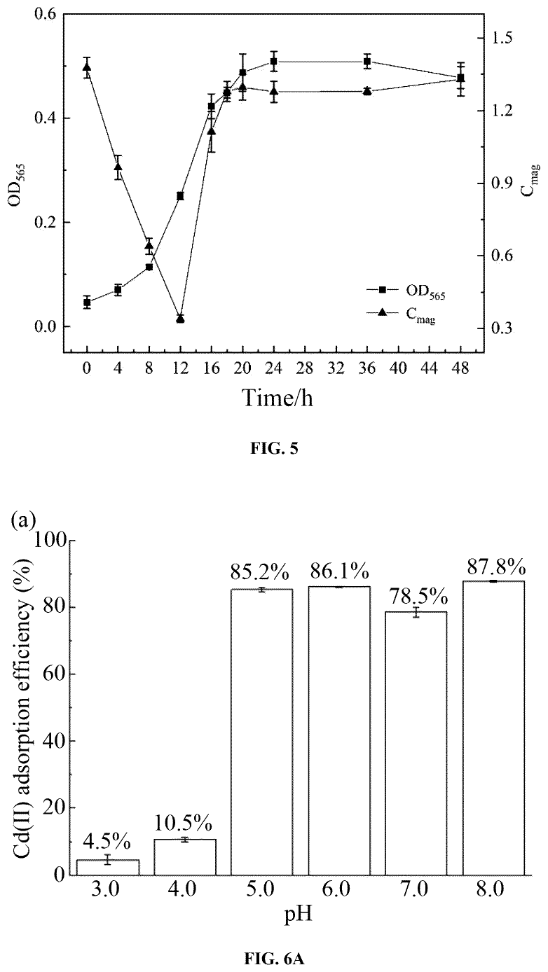

As shown in FIG. 5, the strain growth curve showed that the Magnetospirillum gryphiswaldense strain MSR-1 had a shorter stagnation period in the early growth stage, indicating that the bacterium showed better activity. After 8 h of growth, the strain entered the logarithmic growth phase and reached the growth plateau after 24 h, at which time the OD 565 nm remained at around 0.5. When the time for culture was extended to 48 h, OD₅₆₅ₙₘ slightly decreased to around 0.4. As for the synthesis dynamics of magnetosomes, the C_{mag} value of the Magnetospirillum gryphiswaldense strain MSR-1 showed a trend of decreasing and then increasing. The C_{mag} value of bacteria at the initial point was higher at 1.2. The C_{mag} value dropped from 1.2 to 0.6 in the early stage of culture, which might be because MSR-1 began to grow but had not yet synthesized a large number of magnetosomes in the early stage of growth. At 18 h of culture, the C_{mag} of MSR-1 rapidly increased to 1.3 and remained relatively stable during the subsequent culture, such that an optimal time for culture the strain was determined to be 24 h.

### Example 3

Removal of Cadmium from Cadmium Polluted Wastewater by Magnetospirillum gryphiswaldense Strain MSR-1 and Influencing Factors Thereof

Different concentrations of cadmium chloride (CdCl₂) solutions were used to simulate cadmium polluted industrial wastewater, and batch experiments were conducted to study the effects of different solution pH values, initial Cd²⁺ concentrations, bacterial biomass, and temperature on the adsorption and removal of Cd²⁺in wastewater by the Magnetospirillum gryphiswaldense strain MSR-1. The setting was specifically as follows:

The initial Cd²⁺ concentration of 1 mg/L, MSR-1 biomass concentration of 3.33 g/L, and temperature of 30° C. were kept constant, and experiments were conducted at different pH values (3.0, 4.0, 5.0, 6.0, 7.0, and 8.0) to investigate the effect of pH value. The pH value of 6.0, MSR-1 biomass concentration of 3.33 g/L, and temperature of 30° C. were kept constant, and experiments were conducted at different initial Cd²⁺ concentrations (0.5, 1, 2, 5, 10, 15, and 20 mg/L) to investigate the effect of initial Cd²⁺ concentration. The pH value of 6.0, initial Cd²⁺ concentration of 5 mg/L, and temperature of 30° C. were kept constant, and experiments were conducted at different MSR-1 biomass concentrations (1.67, 3.33, 6.67, and 10 g/L) to investigate the effect of bacterial biomass. The pH value of 6.0, initial Cd²⁺ concentration of 5 mg/L, and MSR-1 biomass concentration of 3.33 g/L were kept constant, and the experiments were conducted in a shaker at different temperatures (20° C., 25° C., 30° C., and 37° C.) to investigate the effect of temperature. The adsorption experiment was conducted in a 50 mL centrifuge tube containing 30 mL of Cd²⁺ solution. For each experimental group inoculated with strain, a control group was set by adding an equal volume of 0.9% sterile NaCl solution added, and each treatment was repeated 3 times. The Cd mass concentration was determined by ICP-MS, and the pollution reduction and removal effect of magnetotactic bacteria on cadmium polluted water was studied under the optimal conditions.

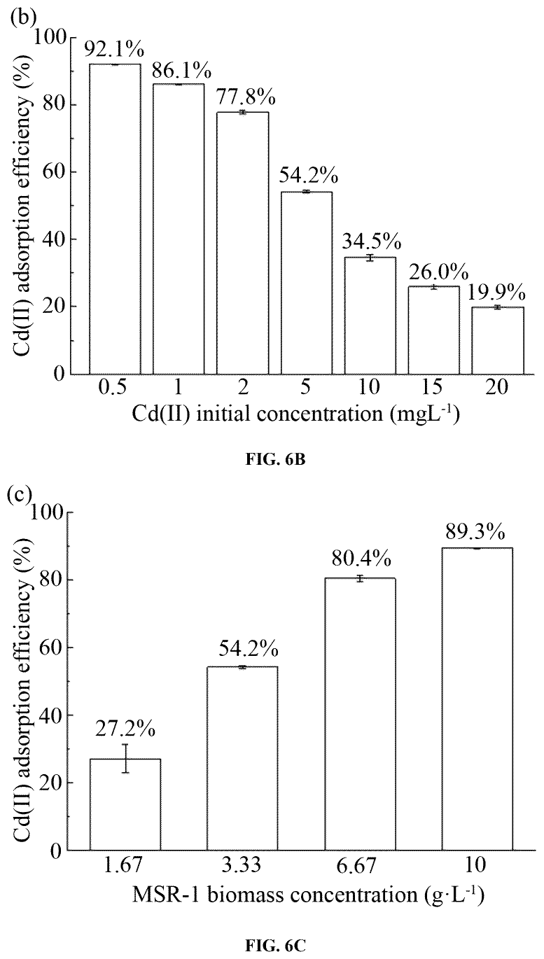

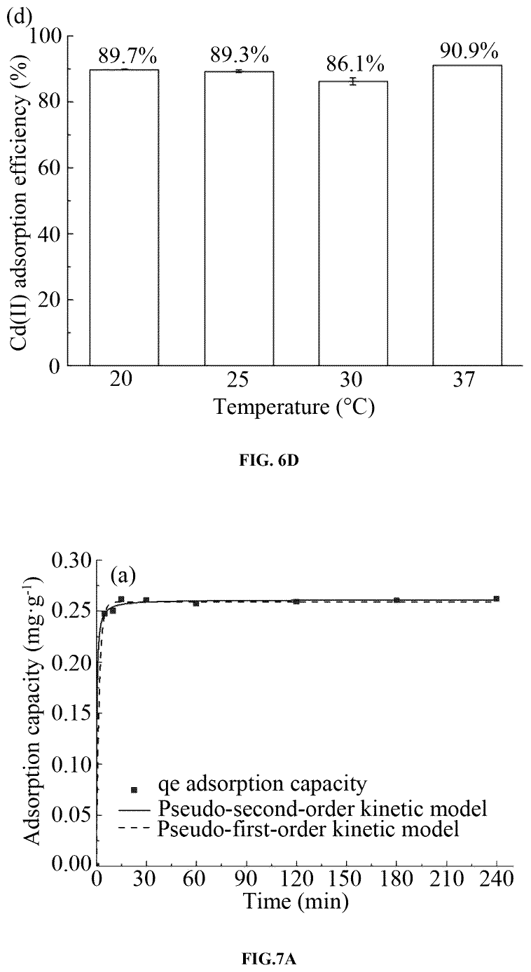

As shown in FIGS. 6A-6D, the adsorption efficiency of Cd by Magnetospirillum gryphiswaldense strain MSR-1 was mainly affected by pH value, initial Cd concentration, and bacterial biomass. Under the optimal reaction conditions (pH=8.0, temperature 30° C., adsorption time 1 h, bacterial biomass 3.33 g/L), the adsorption efficiency of the Magnetospirillum gryphiswaldense strain MSR-1 could reach 87.8% in a solution system with a Cd content of 1.0 mg/L.

### Example 4

### Adsorption Thermodynamics and Adsorption Isotherms of Cd²⁺by Strain MSR-1

The solution pH value was set to 6.0 and the initial Cd²⁺ concentration was set to 1 mg/L. 0.1 g of MSR-1 was added into a 100 mL Erlenmeyer flask containing 30 mL of Cd²⁺solution (MSR-1 biomass concentration was 3.33 g/L), and a corresponding volume of 0.9% sterile NaCl solution was added as a control group. The Erlenmeyer flask was cultured in a shaker at 180 r/min and 30° C., and samples were taken at 5, 10, 15, 30, 60, 120, 180, and 240 min separately after the reaction. The bacterial solution was centrifuged at 10,000 rpm for 3 min, and the supernatant was filtered through a 0.22 μm filter membrane, and the cadmium mass concentration was determined using ICP-MS (PE Nexion 2000, USA). Kinetic model fitting was conducted for the adsorption, where pseudo-first-order kinetic model (1) and pseudo-second-order kinetic model (2) used were as follows:

$${q}_{t} = {q}_{e} \times ( 1 - {exp}^{( - {k}_{1} t )} ) \qquad ( 1 )$$

$${q}_{t} = {k}_{2} {tq}_{e}^{2} / ( 1 + {k}_{2} {tq}_{e} ) \qquad ( 2 )$$

qₑ represented the theoretical adsorption capacity at equilibrium (mg/g), t represented the adsorption reaction time (min), qt represented the adsorption capacity at time t (mg/g), k₁ represented the pseudo-first-order adsorption rate constant (min⁻¹), and k₂ represented the pseudo-second-order adsorption rate constant (g/(mg min)).

Experimental settings for adsorption isotherms: the pH value 6.0, MSR-1 biomass concentration 3.33 g/L, and temperature 30° C. were kept constant, and experiments were conducted at different initial Cd²⁺ concentrations (0.5, 1, 2, 5, 10, 15, and 20 mg/L). The adsorption of MSR-1 to Cd²⁺was fitted using a Langmuir isotherm adsorption model (3) and a Freundlich isotherm adsorption model (4):

$${q}_{e} = {q}_{m} \times {k}_{L} {C}_{e} / ( 1 + {k}_{L} \times {C}_{e} ) \qquad ( 3 )$$

$${q}_{e} = {k}_{F} \times {C}_{e}^{( 1 / n )} \qquad ( 4 )$$

qₑ represented the theoretical adsorption capacity at equilibrium (mg/g), Cₑ represented the concentration of Cd²⁺in the solution at adsorption equilibrium (mg/L), qₘ represented the saturated adsorption capacity of the monolayer (mg/g), k_{L} represented the Langmuir equilibrium constant, and k_{F} and n represented the Freundlich constant.

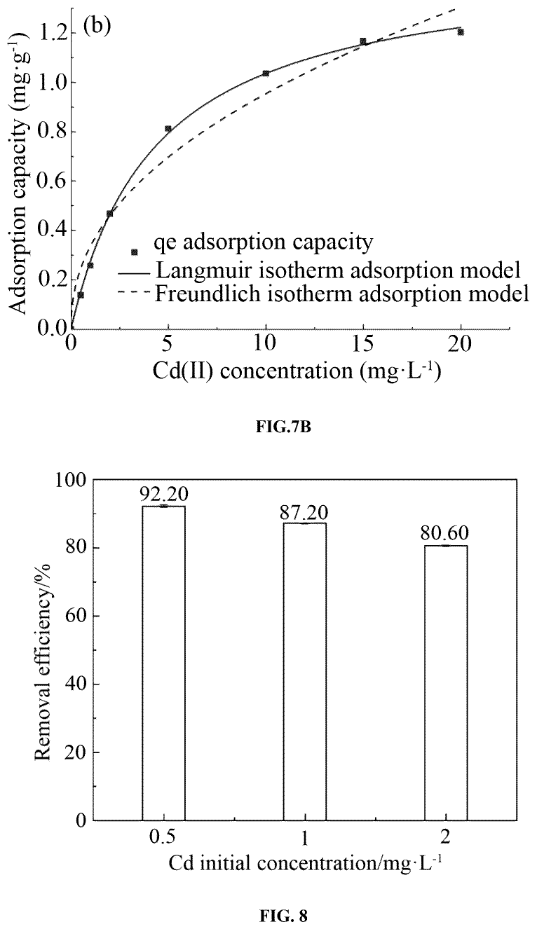

As shown in FIGS. 7A-7D, the adsorption of MSR-1 to Cd²⁺was rapid. The adsorption capacity of Cd²⁺by MSR-1 reached 94.3% of the equilibrium adsorption capacity 5 min after the reaction started, and reached equilibrium 15 min after the reaction started.

The adsorption capacity of Cd²⁺by MSR-1 showed an obvious upward trend with the increase of Cd²⁺ concentration. When the Cd²⁺ concentration was 20 mg/L, the adsorption capacity of MSR-1 reached 1.20 mg/g.

### Example 5

Effect of the Magnetospirillum gryphiswaldense Strain MSR-1 on Removal of Cadmium from Cadmium Polluted Water

Simulated cadmium-containing industrial wastewater (Cd²⁺-containing wastewater with different mass concentrations prepared by CdCl₂, with mass concentrations of 0.5, 1, and 2 mg/L, respectively) was used as an experimental sample for testing. The pH value was adjusted to 6.0. The activated Magnetospirillum gryphiswaldense strain MSR-1 was inoculated into the optimized medium in Example 2 and cultured in a shaker at 30° C. under 110 r/min for 24 h, and Magnetospirillum gryphiswaldense strain MSR-1 after amplification culture was added into the polluted water at 3.3 g/L. The polluted water of each group was stirred evenly after being mixed with the strain, and cultured in a shaker at 30° C. under 180 r/min for 1 h to separate the sewage from the bacterial cells to realize the removal of heavy metal cadmium in the sewage. The cadmium content of each group of water after separation was tested, with 3 replicates set for each treatment group, and the cadmium mass concentration was determined by ICP-MS.

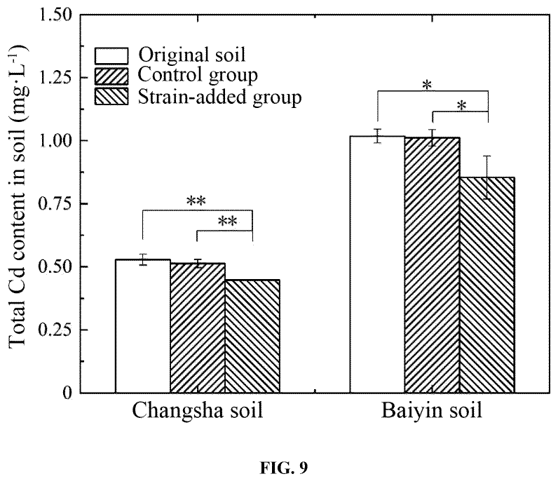

As shown in FIG. 8, the Magnetospirillum gryphiswaldense strain MSR-1 had a desirable adsorption capacity for cadmium in simulated cadmium-containing industrial wastewater with different mass concentrations, and the adsorption efficiencies of cadmium in simulated cadmium-containing industrial wastewater with different mass concentrations (at mass concentrations 0.5, 1, and 2 mg/L, respectively) were 92.2%, 87.2%, and 80.6%, respectively, showing a certain potential for cadmium removal, reduction, and remediation.

### Example 6

Effect of the Magnetospirillum gryphiswaldense Strain MSR-1 on Removal of Cadmium from Cadmium Polluted Soil

Acidic soil (Changsha soil, total Cd content 0.51 mg/kg, pH=5.28) and alkaline soil (Baiyin soil, total Cd content 1.01 mg/kg, pH=8.84) were used as tested soil samples, and the Magnetospirillum gryphiswaldense strain MSR-1 was used as the tested strain. The two tested soil samples were ground to pass through a 0.85 mm nylon sieve, and a certain amount of the tested soil samples were weighed and added into a 50 mL centrifuge tube. Water was added at a mass ratio of soil to water of 1: (8-12) and stirred evenly to obtain two groups of water-soil mixture samples. The activated Magnetospirillum gryphiswaldense strain MSR-1 was conducted to amplification culture, and 0.15 g of the Magnetospirillum gryphiswaldense strain MSR-1 was resuspended in 1.0 mL of NaCl (0.9%) solution and then added into the two groups of water-soil mixture samples to obtain an experimental group. An equal volume of NaCl (0.9%) solution without bacteria was added as a control group. The centrifuge tube was placed in a shaker for oscillation and reaction, then taken out and allowed to stand for 1 h. A magnet was attached to a side wall of the centrifuge tube and allowed to stand for 24 h. The supernatant and soil were separated by a pipette without disturbing the magnetically enriched bacterial cells on the side wall, and the supernatant, magnetically enriched bacterial cells, and the lower soil were separately preserved. The air-dried soil was digested and separated using a fully automatic graphite digestion instrument, and the obtained bacterial cells were digested using a graphite furnace digestion instrument. The above digestion solution and supernatant were filtered through a 0.45 μm filter membrane and then the cadmium content in the soil was determined by ICP-MS, while standard substances and blank samples were set for quality control.

As shown in FIG. 9, the change in total cadmium in the soil after treatment and separation of the Magnetospirillum gryphiswaldense strain MSR-1 proved that after MSR-1 reacted with Changsha soil and Baiyin soil and was magnetically separated, the total Cd content of Changsha and Baiyin soils decreased by 12.7% and 15.6%, respectively, showing a certain potential remediation for cadmium removal and reduction.

The above descriptions are merely preferred embodiments of the present disclosure. It should be noted that a person of ordinary skill in the art may further make several improvements and modifications without departing from the principle of the present disclosure, but such improvements and modifications should be deemed as falling within the protection scope of the present disclosure

What is claimed is:

1. A Magnetospirillum gryphiswaldense strain MSR-1, wherein the Magnetospirillum gryphiswaldense strain MSR-1 is deposited in China Center for Type Culture Collection with a deposit number of CCTCC NO: M 20242494.

2. A medium for culturing the Magnetospirillum gryphiswaldense strain MSR-1 according to claim 1, comprising a carbon source selected from the group consisting of sodium lactate, sodium acetate, tartaric acid, and sodium lactate, a nitrogen source selected from the group consisting of ammonium chloride, sodium nitrate, and ammonium sulfate, an iron source selected from the group consisting of ferrous sulfate, ferric citrate, and ferric quinate, and a reducing agent selected from the group consisting of sodium thioglycolate, ascorbic acid, and cysteine.

3. The medium according to claim 2, comprising 2 g/L to 5 g/L of the carbon source, 0.2 g/L to 0.5 g/L of the nitrogen source, 0.005 g/L to 0.02 g/L of the iron source, and 0.03 g/L to 0.06 g/L of the reducing agent.

4. The medium according to claim 2, comprising 3 g/L to 4 g/L of the sodium lactate, 0.35 g/L to 0.50 g/L of the ammonium chloride, 0.010 g/L to 0.015 g/L of the ferric citrate, and 0.045 g/L to 0.060 g/L of the sodium thioglycolate.

5. A culture method of Magnetospirillum gryphiswaldense strain MSR-1, comprising the following steps: inoculating an activated Magnetospirillum gryphiswaldense strain MSR-1 into the medium according to claim 4, and then culturing at 25° C. to 35° C. under 90 r/min to 120 r/min for 18 h to 48 h;
wherein the Magnetospirillum gryphiswaldense strain MSR-1 is deposited in China Center for Type Culture Collection with a deposit number of CCTCC NO: M 20242494.

6. The culture method of the Magnetospirillum gryphiswaldense strain MSR-1 according to claim 5, wherein the culturing is conducted for 20 h to 36 h.

7. A preparation product, comprising the Magnetospirillum gryphiswaldense strain MSR-1 according to claim 1.

8. A method for remediation of cadmium pollution, comprising using the Magnetospirillum gryphiswaldense strain MSR-1 according to claim 1, wherein the cadmium pollution is cadmium pollution in water and/or soil.

9. The method according to claim 8, wherein the water or the soil is adjusted to a pH value of 5.0 to 8.0, and an initial cadmium concentration is no more than 2 mg/L during the remediation.

10. The method according to claim 8, wherein the Magnetospirillum gryphiswaldense strain MSR-1 is inoculated into the water at 3 g/L to 10 g/L; and
the soil is mixed with water to obtain a soil-water mixture, and the Magnetospirillum gryphiswaldense strain MSR-1 is inoculated into the soil-water mixture at 3% to 5% of a soil mass.

11. A method for remediation of cadmium pollution, comprising using the preparation product according to claim 7, wherein the cadmium pollution is cadmium pollution in water and/or soil.
